SEQUENCE LISTING (5 sequences)

SEQ ID NO: 1 - DNA, 25 bp, Artificial Sequence
  3P Primer for Tobacco
aaaacccgtcctcagttcggattgc

SEQ ID NO: 2 - DNA, 24 bp, Artificial Sequence
  Primer 16SF for Lettuce
cagcagccgcggtaatacagagga

SEQ ID NO: 3 - DNA, 25 bp, Artificial Sequence
  Primer 3M
ccgcgttgtttcatcaagccttacg

SEQ ID NO: 4 - DNA, 24 bp, Artificial Sequence
  Primer 5P
ctgtagaagtcaccattgttgtgc

SEQ ID NO: 5 - DNA, 25 bp, Artificial Sequence
  Primer 2M
tgactgcccaacctgagagcggaca

(19) United States
(12) Patent Application Publication
(10) Pub. No.: US 2015/0196627 A1
(43) Pub. Date: Jul. 16, 2015

(54) PLASTID-EXPRESSED MYCOBACTERIUM TUBERCULOSIS VACCINE ANTIGENS ESAT-6 AND MTB72F FUSED TO CHOLERA TOXIN B SUBUNIT

(71) Applicant: University of Central Florida Research Foundation, Inc., Orlando, FL (US)

(72) Inventor: Henry Daniell, Philadelphia, PA (US)

(21) Appl. No.: 14/353,998

(22) PCT Filed: Oct. 24, 2012
(86) PCT No.: PCT/US2012/061611

§ 371 Date: Apr. 24, 2014

Related U.S. Application Data
(60) Provisional application No. 61/550,834, filed on Oct. 24, 2011.

(51) Int. Cl. A61K 39/04; C07K 14/35

(52) U.S. Cl. CPC A61K 39/04; C07K 14/35

(57) ABSTRACT

Tuberculosis (TB) is caused by Mycobacterium tuberculosis and is one of the leading reasons for death by an infectious bacterial pathogen. Disclosed herein are compositions and methods of using same related to chloroplast expressed TB antigens. Also disclosed is the bioencapsulation of the TB antigens that enables the oral administration of the composition while preserving immunizing efficacy.

### CROSS-REFERENCE TO RELATED APPLICATIONS

This application is related to U.S. Provisional Application No. 61/550,834, filed Oct. 24, 2011, to which priority is claimed under 35 USC 119. The entire disclosure of this provisional application is incorporated herein by reference.

### STATEMENT OF FEDERAL FUNDED RESEARCH

The work disclosed herein was supported at least in part by the National Institute of Health, under grant number NIH R01 GM 63879. The U.S. government has rights in the invention.

### BACKGROUND

Tuberculosis (TB) caused by Mycobacterium tuberculosis (MTB), is one of the leading bacterial infections that is re-emerging due to drug resistant strains worldwide. The World Health Organization (WHO) estimates the global burden of TB in 2009 to be 9.8 million incident cases with multi drug resistant TB growing at a rate of half a million cases every year (WHO 2010). TB is also the leading cause of death in HIV-infected patients as immunosuppression increases risk of reactivation of TB. Bacillus Calmette Guerin (BCG), an attenuated strain of Mycobacterium bovis is the only available licensed vaccine against TB. Many trials that evaluated BCG on the basis of protective immunity and age of vaccination have been inconsistent and variable, ranging from 0 to 80% efficacy. (Colditz et al. 1994; Brewer et al. 1995; Xing et al. 2006). BCG does not prevent the establishment of latent TB or reactivation of pulmonary disease in adults (Andersen 2007). To remedy this, research groups are engaged in developing more efficient anti-TB vaccines which may have the potential to replace BCG as a primary TB vaccine or act as an effective boosting vaccine (Derrick et al. 2004; Andersen 2007; Doherty et al. 2007; Coler et al. 2009). Compared to attenuated live TB vaccines, subunit vaccines offer several advantages including safety, efficacy and they are better suited for standardization (Agger et al. 2001; Tsenova et al. 2006). On the other hand, limitations include poor immunogenicity of purified antigens and restriction in the number of antigens exposed. This makes an immunostimulatory component all the more essential in an effective vaccine.

Many different fractions of MTB have been proposed as subunit vaccine candidates including surface components and secreted proteins. Some of the promising antigens are ESAT-6, Ag85B, MTB72F etc. Any mycobacterial antigen which could activate both CD4 & CD8 T-cells and imparts protective immunity is the ideal candidate for subunit vaccination against TB. Antigenic proteins actively secreted during the early phase of growth of MTB are best suited as potential candidates for subunit vaccines (Brodin et al. 2004).

ESAT-6 (6 kDa early secretory antigenic target) is one such promising vaccine antigen candidate that can strongly elicit a specific T-cell response (Brandt et al. 2000). ESAT-6 has been established to be present in the RD-1 region in all virulent strains of MTB but its also striking that its absent in the attenuated BCG vaccine strain (Andersen et al. 1995). Hence ESAT-6 could prove to be one of the components essential to treat this complex disease. It is reported to induce production of gamma interferon (IFN-γ), a marker for protective immune response (Agger et al. 2001; Kumar et al. 2010) with protective immunity comparable to BCG (Brandt et al. 2000). The vaccines based on ESAT-6 antigen in combination with another mycobacterial antigen Ag85B have entered human clinical trials (van Dissel et al. 2010).

Another attractive vaccine antigen candidate is Mtb72F, a recombinant fusion polyprotein from two known TB antigens Mtb32 and Mtb39. Mice immunization with the recombinant protein Mtb72F, formulated in two different adjuvant systems namely AS01B and AS02A (GlaxoSmithKline Biologicals proprietary adjuvants), resulted in the induction of strong immune response (Tsenova et al. 2006). It was also reported that vaccinated mice were protected against aerosol challenge with a virulent strain of MTB (Skeiky et al. 2004). Vaccination with Mtb72F formulated in AS02A or AS01B was also protective against central nervous system (CNS) challenge with Mycobacterium tuberculosis H37Rv (Tsenova et al. 2006).

Mycobacterial antigens as subunit vaccines have been targeted by different delivery systems including recombinant viral vector system (Sereinig et al. 2006), recombinant bacterial vector system (Triccas 2010), lipoglycan-protein conjugate system (Hamasur et al. 2003). Disclosed herein are embodiments related to delivering tuberculosis antigens to the gut associated lymphoid tissue (GALT)—an integral part of the mucosal immune system. TB, being a respiratory disease, subunit vaccination targeting the mucosa is well disposed to initiate both mucosal and systemic immune response. In order to survive the extreme physiological conditions of the gut, strategies have to be employed to protect the antigen and optimize dosage conditions to ensure antigen uptake.

### BRIEF DESCRIPTION OF THE DRAWINGS

FIG. 1: Vector construction for chloroplast transformation. (a) The pLD-CTB-ESAT6 vector. (b) The pLD-CTB-MTB72F vector. (e) pLS-CTB-ESAT6 vector. The primers 3P/3M or 16SF/3M and 5P/2M anneal on the aadA and chloroplast flanking sequences respectively to determine Integration of the transgene cassette(s) and the gene of interest into the chloroplast genome. The fragment sizes between the two Hind III sites represent the expected products from the digestion of plants transformed with pLD-CTB-ESAT6 & pLD-CTB-MTB72F. (c) & (d) Schematic diagram of expected products from digestion of the untransformed tobacco and lettuce chloroplast genome respectively for the flanking sequence hybridization probe.

FIG. 2: Confirmation of site specific transgene Integration in Tₒ transplastomic plants by PCR and Southern blot analysis. (a) & (b) PCR analysis of tobacco transplastomic plants using 3P/3M primers. (c) PCR analysis of lettuce transplastomic plants using 16sf/3M primers. (d) & (e) PCR analysis of tobacco transplastomic plants using 5P/2M primers. (f) PCR analysis of lettuce transplastomic plants using 5P/2M primers. Southern blot analysis of (g) tobacco CTB-ESAT6 (CTB-ESAT6 (Nt)) homoplasmy transplastomic plants, (h) tobacco CTB-MTB2F transplastomic plants and (i) lettuce CTB-ESAT6 (CTB-ESAT6 (Ls)) homoplasmy transplastomic plants (N, untransformed plant; Number 1 to 9, transformed plants; P, positive control; M, DNA ladder).

FIG. 3: Anti CTB Western blot analysis demonstrating expression of the fusion protein in transplastomic plants. (a), (b) & (c) Tobacco CTB-ESAT6 (Nt), tobacco CTB-MTB72F and lettuce CTB-ESAT6 (Ls) transplastomic plants. (d), (e) & (f) Different protein extraction fractions from tobacco CTB-ESAT6 (Nt), tobacco CTB-MTB72F and lettuce CTB-ESAT6 (Ls) transplastomic leaf tissue (N, Untransformed; H, homogenate; S, supernatant; P, pellet; 1 to 4, transplastomic plants; C, purified bacterial CTB standard; C1, C2, C3 & C4: CTB standards 25, 50, 75, 100 ng for densitometry).

FIG. 4: Quantification of CTB-ESAT6 (Nt) and CTB-MTB72F (Nt) expressed in T₀ tobacco transplastomic plants at different developmental stages and harvesting time quantified by ELISA

(a) Expression levels of tobacco CTB-ESAT6 (Nt) in percent TSP in different age plants for different time points under the normal growth condition. (b) Expression levels of tobacco CTB-MTB72F (Nt) in percent TSP in young, mature and old leaves collected from the transplastomic plants at 12 p.m.

FIG. 5: Quantification of CTB-ESAT6 (Ls) in T₀ lettuce transplastomic plants at different developmental stages and harvesting time by densitometry. (a) Expression levels of lettuce CTB-ESAT6 (Ls) in percent TP at different developmental stages. (b) Expression levels at different harvesting time points. (1 to 3: transplastomic lines). (c) Standard curve was established using 25, 50 and 75 ng of purified CTB for densitometry. (d) Immunoblot analysis of comparison of fresh weight and lyophilized material. Fresh leaf and Lyophilized: Equal amount of leaf material in equal volume of protein extraction buffers. Equal volume loaded with dilutions of 1×, 10×, 20×; CTB: CTB standards 50 ng;

FIG. 6: Analysis of affinity purified fractions of CTB-ESAT6. a) Immunoblot to confirm the presence of antigen with CTB antibody. S1, S2, S3: CTB standard samples (12.5, 25, 37.5 ng); E1, E2, E3: elution fractions (100, 50, 25 ng) respectively; B4IP—CTB-ESAT6 protein before purification (10 μg); WT—wildtype lettuce total leaf protein (10 μg). (b) Silver staining after affinity purification of CTB-ESAT6. C1, C2, C3, C4: purified CTB standards (100, 200, 300, 400 ng); After affinity purification, elution fractions of CTB-ESAT6 (LS)—E1 (400 ng), E2 (200 ng). M-protein standard marker 0.4 μl.

FIG. 7: Functional analysis of CTB-ESAT6 fusion protein. (a), (b) Ganglioside GM1 ELISA binding assay. (a) Functionality of CTB was confirmed based on its binding affinity to Ganglioside GM1 (E1, E2—CTB-ESAT6 (Ls) total protein (15 μg); WT—Wild type lettuce leaf protein (15 μg); CTB—purified CTB standard—50 ng; BSA—50 ng. (b) GM1 ELISA comparison between lyophilized and fresh tissue, loaded at equal concentrations of TSP (15 μg) with decreasing dilutions gave similar trend. Purified CTB—10 ng. (c) Hemolysis assay to determine functionality of ESAT-6 in partially purified fusion protein CTB-ESAT6 (Ls). H₂0—distilled water; E1—CTB-ESAT6—partially purified protein 40 μg/ml; E2—CTB-ESAT6—partially purified protein 20 μg/ml; PBS—control; Elution/sol buffer—elution buffer used for protein elution/solubilisation; Nonsol E1—CTB-ESAT6 (Ls) before solubilisation. CTB—purified protein 40 μg/ml

### DETAILED DESCRIPTION

The disclosure includes description of methods of making and using chloroplast expressed TB antigens. Also, the disclosure includes description of temperature stable, freeze-dried plant materials the contain plastid encapsulated TB antigens that have a significant shelf-live and retain effectiveness for weeks.

Cholera toxin B subunit is a well researched mucosal adjuvant that has been reported as carrier molecules for mucosal immune responses and oral tolerance (Langridge et al. 2010). It is believed that CTB fused with TB antigens could potentiate systemic and mucosal immune response. Plant vaccines are highly efficient delivery vehicles as they are capable of transporting large amount of antigen in an encapsulated form. In animal trials, many plant oral vaccines expressing foreign proteins fused to CTB have shown to be protected against degradation by stomach enzymes and offer protective immunity against disease states (Ruhlman et al. 2007; Arlen et al. 2008; Davoodi-Semiromi et al. 2010).

Recombinant ESAT-6 has been expressed in transient plant production systems such as potato virus X vector based system (PVX), tobacco mosaic virus vector based system, agrobacterium mediated transformation (Rigano et al. 2004; Zelada et al. 2006; Dorokhov et al. 2007). Provided herein is the first development and demonstration ESAT-6 or MTB72F fused with CTB was expressed in tobacco as a model system for production, followed by development of CTB-ESAT6 lettuce chloroplasts to explore the possibility of chloroplast as an efficient bioreactor for oral delivery of vaccine antigens against TB. The expression levels of TB antigens were analyzed using western blot analysis. Lyophilization was performed on lettuce leaves for investigating stability and storage. GM-1 binding ELISA assay confirmed binding affinity of CTB-ESAT6 to GM-1 receptor. Hemolysis assay demonstrated dose dependent hemolytic activity of CTB-ESAT6 in red blood cell membranes.

According to one embodiment, provided herein is a plant cell of a plant, wherein said plant cell comprises chloroplasts transformed to express CTB-ESAT-6 or CTB-MTB72F. In a specific embodiment, the plant cell is edible.

In another embodiment, disclosed herein is an orally-administrable composition comprising CTB-ESAT-6 or CTB-MTB72F expressed in a chloroplast; and, optionally, rubisco. The chloroplast may be from an edible plant. Examples of edible plants include, but are not limited to, Lactuca sativa, carrot, tomato, strawberry, citrus, or banana.

According to another embodiment, disclosed herein is a sample of CTB-ESAT-6 or CTB-MTB72F bioencapsulated in chloroplasts of a plant cell. In a specific aspect, the plant cell is from an edible plant. In another related embodiment, the plant cell is homoplasmic with respect to plant plastids transformed to express said CTB-ESAT-6 or CTB-MTB72F.

In another embodiment, disclosed herein is an orally-administrable composition comprising CTB-ESAT-6 or CTB-Mtb72F expressed in a chloroplast; and optionally rubisco. The chloroplast may be from an edible plant. Examples of edible plants include plants that are edible without cooking, i.e., edible without the need to be subjected to heat exceeding 120 degrees Fahrenheit for more than 5 min. Examples of such edible plants include, but are not limited to, Lactuca sativa (lettuce), apple, berries such as strawberries and raspberries, citrus fruits, tomato, banana, carrot, celery, cauliflower; broccoli, collard greens, cucumber, muskmelon, watermelon, pepper, pear, grape, peach, radish and kale. In a specific embodiment, the edible plant is Lactuca sativa.

According to another embodiment, disclosed herein is a Lactuca sativa plant plastid comprising a plastid genome transformed with a heterologous DNA coding sequence encoding a CTB-ESAT-6 or CTB-MTB72F, and integrated into said plastid genome such that said CTB-ESAT-6 or CTB-Mtb72F is expressed in and present in said plastid. In a related embodiment, the Lactuca sativa plant cell is homoplasmic with respect to plastids transformed to express a CTB-ESAT-6 or CTB-MTB72F.

In a further embodiment, the invention relates to a method of vaccinating a subject against TB comprising administering to said subject a composition comprising a CTB-ESAT-6 or CTB-MTB72F polypeptide expressed in a chloroplast in a plant and, optionally, a plant remnant. The plant remnant may be rubisco.

An additional embodiment relates to a composition for retarding the development of or treating diabetes comprising a therapeutically effective amount of a CTB-ESAT-6 or CTB-MTB72F polypeptide and a plant remnant from Lactuca sativa.

In yet a further embodiment, disclosed herein is a plastid transformation and expression vector for transformation of Lactuca sativa plastid, said vector comprises an expression cassette comprising, as operably linked components in the 5′ to the 3′ direction of translation, a promoter operative in said plastid, a selectable marker sequence, a heterologous polynucleotide sequence coding a CTB-ESAT-6 or CTB-MTB72F protein, transcription termination functional in said plastid, and flanking each side of the expression cassette, flanking DNA sequences which are homologous to a DNA sequence of the target plastid genome, whereby stable integration of the heterologous coding sequence into the plastid genome of the target plant is facilitated through homologous recombination of the flanking sequence with the homologous sequences in the target plastid genome. In a related embodiment, the plastid is selected from the group consisting of chloroplasts, chromoplasts, amyloplasts, proplastide, leucoplasts and etioplasts. In another related embodiment, the selectable marker sequence is an antibiotic-free selectable marker.

Plants stably transformed to include a plastid stably transformed with vectors described herein, or the progeny thereof, including seeds is disclosed.

### EXAMPLES

### Example 1

### Construction of Tobacco and Lettuce Chloroplast Transformation Vectors Containing TB Antigens

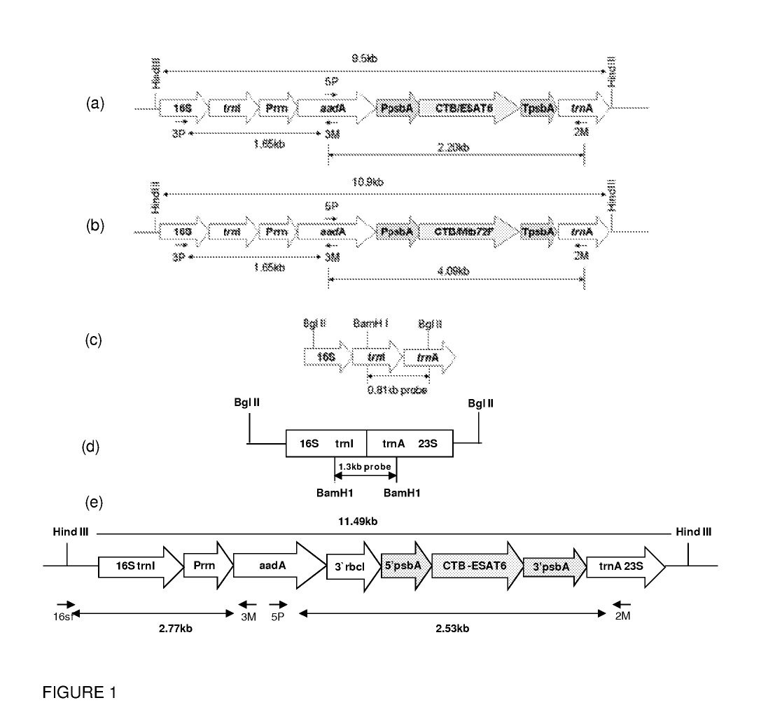

Tobacco chloroplast vectors pLD-CTB-ESAT6 and pLD-CTB-MTB72F were constructed with CTB-ESAT6 and CTB-MTB72F coding sequence respectively (FIGS. 1a & b). The lettuce chloroplast vector pLS-CTB-ESAT6 harboring CTB-ESAT6 was also created (FIG. 1e). Both CTB-ESAT6 and CTB-MTB72F contained GPGP (Gly Pro Gly Pro) hinge in the middle of fusion proteins to assist in correct folding of each protein by lowering the steric hindrance. The tobacco vectors contained homologous flanking sequences 16S/trnI and trnA (FIG. 1c) whereas lettuce vector had longer flanking sequences 16S/trnI and trnA/23S (FIG. 1d) to facilitate recombination into the native chloroplast genome. The fusion gene cassettes were regulated by endogenous psbA promoter and 5□ untranslated region (UTR) to achieve higher levels of expression due to the presence of multiple ribosome binding sites ((Fernandez-San Millan et al. 2003; Ruhlman et al. 2010). The psbA 3□ UTR located at the 3□ end of the introduced gene cassette conferred transcript stability (Stern et al. 1987). The endogenous constitutive 16S rRNA promoter (Prrn) was employed to regulate expression of the aadA (aminoglycoside 3′ adenyltransferase) gene with a GGAG ribosome binding site upstream of the start codon AUG (Dhingra et al. 2006) to confer spectinomycin resistance. The final chloroplast transformation vectors pLD-CTB-ESAT6, pLD-CTB-MTB72F and pLS-CTB-ESAT6 were sequenced and used for transformation studies.

### Example 2

### Regeneration of Transplastomic Plants and Confirmation of Site Specific Transgene Integration by PCR Analysis

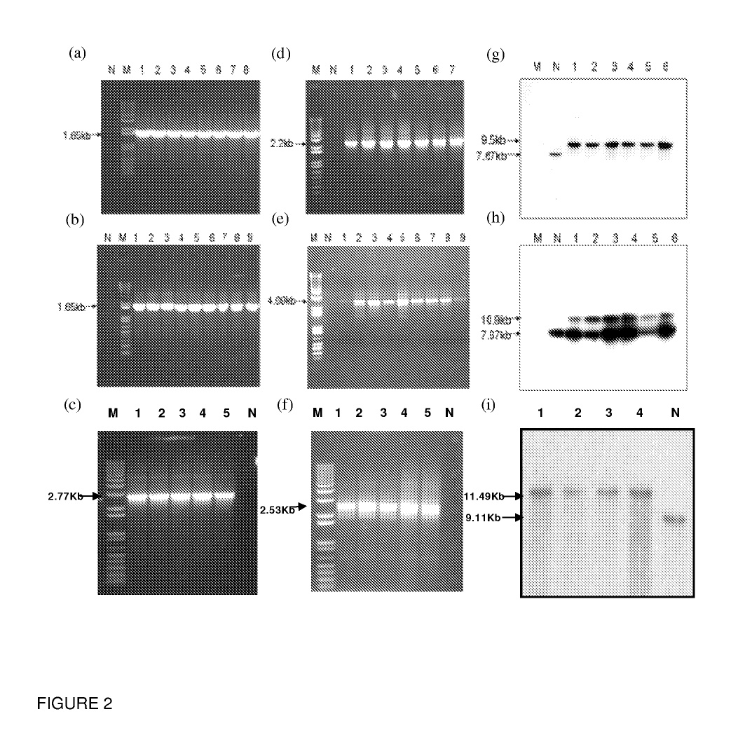

A total of 9 (per 30 bombardments) and 26 (per 40 bombardments) independent spectinomycin resistant tobacco shoots were obtained with pLD-CTB-MTB72F and pLD-CTB-ESAT6 vectors respectively. Four spectinomycin resistant lettuce shoots per forty bombardments were recovered with pLS-CTB-ESAT6 vector coated on gold particles. Site specific transgene integration of independent spectinomycin resistant shoots was verified by polymerase chain reaction (PCR) using 3P/3M and 5P/2M primer pairs in tobacco and 16SF/3M and 5P/2M primer pairs in lettuce. The 3P and 16SF primer anneals to the native chloroplast genome within the 16S rRNA gene whereas 3M primer anneals to the aadA gene (FIGS. 1a, b & e). PCR reaction with 3P/3M primers generated a 1.65 kb PCR product in tobacco CTB-ESAT6 and CTB-MTB72F transplastomic lines (FIGS. 2a & b, Lanes: 1-9) whereas 16SF/3M yielded a 2.77 kb fragment in lettuce CTB-ESAT6 transplastomic lines (FIG. 2c, Lanes: 1-4), which should be obtained only if site specific integration had occurred. Nuclear transformants, mutants and untransformed plants did not show any PCR product (FIGS. 2a, b & c, Lane: N). Nuclear transformants could be distinguished because 3P or 16SF will not anneal and mutants were identified because 3M will not anneal and thus eliminates shoots that have nuclear integration or spontaneous mutation of the 16S rRNA gene. The integration of transgene cassette was further tested by using 5P/2M primer pair. The 5P primer anneals to the aadA gene upstream of CTB-TB antigen fusion gene cassette whereas 2M anneals to the trnA gene (FIGS. 1a, b & e). The 5P/2M primer pair generated a 2.2 kb, 4.09 kb and 2.53 kb PCR product in CTB-ESAT6 tobacco, CTB-MTB72F tobacco and CTB-ESAT6 lettuce transplastomic lines respectively while no amplification was observed in untransformed plants as expected (FIGS. 2d, e & f).

### Example 3

### Evaluation of Homoplasmy in Transplastomic Plants by Southern Blot Analysis

Following PCR analysis, transplastomic plants were subjected to two additional rounds of selection (second and third) to promote homoplasmy. Southern blot analysis was performed to determine homoplasmy or heteroplasmy and to supplement the PCR confirmation of site specific transgene integration. The flanking sequence probe (0.81 kb in tobacco FIG. 1c and 1.13 kb in lettuce FIG. 1d) allowed detection of site-specific integration of the gene cassette into the chloroplast genome as it hybridizes with the trnI and trnA genes. The transformed chloroplast genome digested with HindIII produced fragments of 9.5 kb for pLD-CTB-ESAT6 (FIG. 2g), 10.9 kb for pLD-CTB-MTB72F (FIG. 2h) and 11.49 kb for pLS-CTB-ESAT6 (FIG. 2i) when hybridized with flanking sequence probe. The untransformed tobacco and lettuce chloroplast genome digested with HindIII produced a 7.67 kb fragment and 9.11 kb fragment respectively confirming that these plants lacked foreign genes (FIGS. 2g, h and i). The flanking sequence probe helps us identify if homoplasmy of the chloroplast genome was achieved through more rounds of selection. Absence of 7.67 kb and 9.11 kb fragment in tobacco and lettuce CTB-ESAT6 transplastomic lines respectively confirmed homoplasmy (within the detection limits of Southern blot) and stable integration of foreign genes into the chloroplast genome (FIGS. 2g & i). However, for CTB-MTB72F transplastomic plants, homoplasmy could not be achieved even after six rounds of selection on stringent selection medium containing 500 μg/ml spectinomycin as both 10.9 kb and 7.67 kb fragments corresponding to transformed and untransformed genomes were detected in Southern blot (FIG. 2h). Plants confirmed by Southern analysis were transferred to jiffy pellets and placed in 16 hr light/8 hr dark cycle incubator until they were acclimatized. Later they were transferred to green house where they matured and seeds were collected. Both tobacco and lettuce transplastomic plants showed no visible difference in comparison with the wild type and maintained the normal growth and morphology in our experimental condition (data not shown).

### Example 4

### Analysis of Transgene Segregation

Seeds collected from only T₀ CTB-ESAT6 transplastomic and untransformed lettuce plants were germinated on ½ MS medium with spectinomycin in the same plate. All T1 CTB-ESAT6 lettuce seeds germinated and developed into uniformly green plants. This lack of mendelian segregation of genes confirmed maternal inheritance of transgenes. None of the untransformed seeds germinated on the selection media. Further, after transfer to green house, all T1 plants flowered, set seeds and showed similar phenotype when compared to T₀ and untransformed plants.

### Example 5

### Western Blot Analysis of CTB-ESAT6 and CTB-MTB72F Fusion Protein in Transplastomic Plants

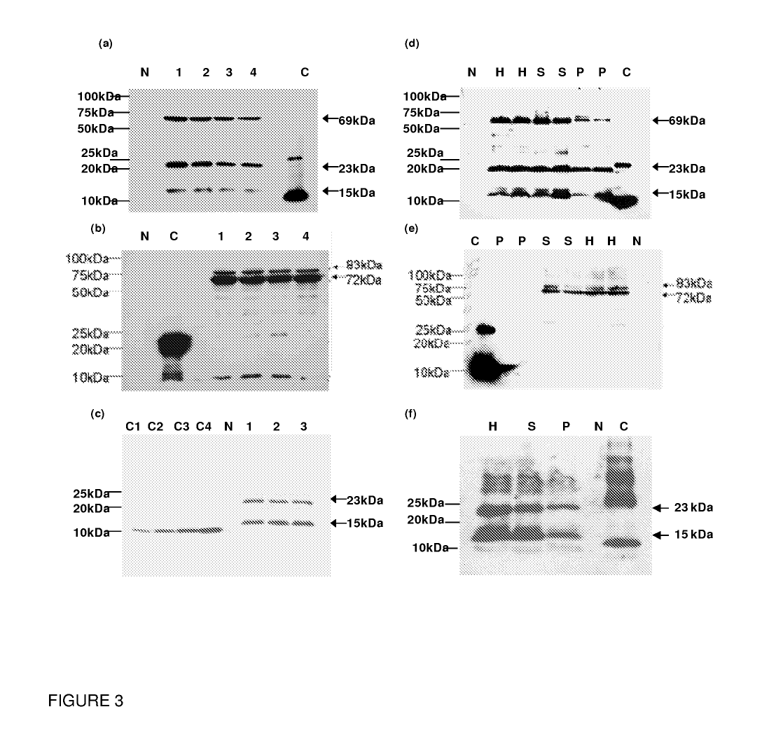

Expression of the fusion proteins ESAT-6 and MTB72F were analyzed by immunoblots with different extraction fractions of leaf extracts using anti-CTB polyclonal antibody. Under reducing conditions, blots probed with anti-CTB polyclonal antibody revealed full-length 23 kDa protein for tobacco CTB-ESAT6 (CTB-ESAT6-Nt, FIG. 3a) which is the expected molecular mass of the fusion protein. Additional 69 kDa protein was also detected indicating the appearance of trimer form of the fusion protein in tobacco chloroplasts (FIG. 3a). In CTB-MTB72F (CTB-MTB72F-Nt) tobacco plants, immunoblots revealed the expected 83 kDa fusion protein besides a ˜72 kDa protein band which might have been formed due to cleavage of full length protein (FIG. 3b). In case of lettuce CTB-ESAT6 (CTB-ESAT6-Ls), the monomeric form of fusion protein corresponding to 23 kDa was observed under reducing conditions (FIG. 3c). Both tobacco and lettuce CTB-ESAT6 plants showed an additional lower band of about 15 kDa with anti-CTB antibody (FIGS. 3a & c). This band might have been formed by proteolytic cleavage of fusion protein due to the presence of putative protease-sensitive sites. Since, the band is larger than CTB protein size (11.6 kDa), cleavage should be in ESAT-6. We performed Peptide Cutter analysis the ExPASy server on ESAT-6 sequence to predict protein cleavage sites {Gasteiger E., 2005 #296}. Peptide Cutter identified several cleavage sites closer to N-terminus of ESAT-6 (˜25 amino acid) indicating a higher probability of cleavage in ESAT-6 protein. Analysis of different extraction fractions of fusion protein CTB-ESAT6 from both tobacco and lettuce indicated that most of the fusion protein existed in soluble form. Some of the fusion protein was also detected in pellet under our experimental conditions (FIGS. 3d & f). On the contrary to CTB-ESAT6, CTB-MTB72F existed only in soluble form and no fusion protein was detected in pellet under our experimental conditions (FIG. 3e).

### Example 6

### Quantification of CTB-ESAT6 and CTB-MTB72F in Tobacco Plants Using ELISA

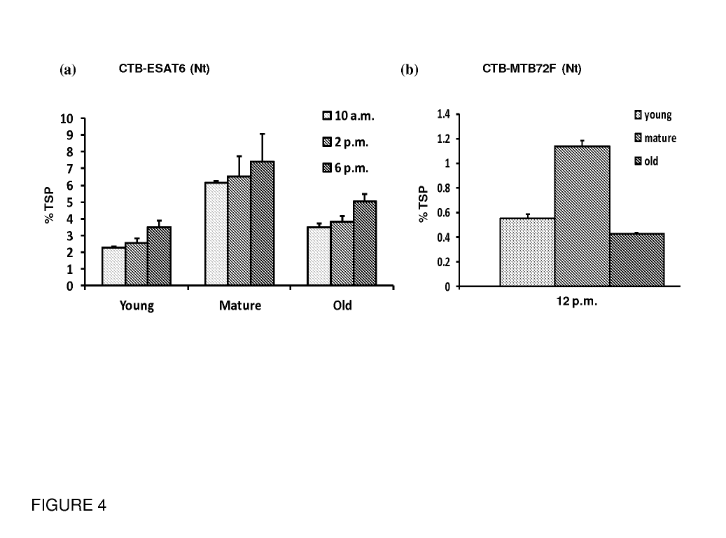

Expression levels of the fusion protein CTB-ESAT6 in transplastomic tobacco plants at different developmental stage and time of leaf harvest were quantified by ELISA using leaf protein extracts prepared from young, mature and old leaves harvested at 10 AM, 2 PM and 6 PM. Under normal illumination conditions (16 h of light and 8 h of dark), the highest expression level of up to 7.5% of total soluble protein (TSP) was observed in mature leaves harvested at 6 PM whereas in young and old leaves the expression levels reached up to 3.4% and 5% of TSP at same harvesting time (FIG. 4a). The large accumulation of CTB-ESAT6(Nt) in mature tobacco leaves probably resulted from higher number of well-developed chloroplasts and the high copy number of chloroplast genomes (Koya et al. 2005). Likewise, decrease in CTB-ESAT6 expression in young and old leaves could be due to less number of underdeveloped chloroplasts and degradation of the proteins during senescence respectively Accumulation of CTB-ESAT6(Nt) was increased over time during the day and reached the highest at 6 PM. (FIG. 4a). This could be attributed to 5□ UTR of the psbA promoter that enhances translation of psbA under light conditions.

Although homoplasmic CTB-MTB72F transgenic plants could not be obtained in our experimental condition, we still quantified the expression of CTB-MTB72F fusion protein in tobacco. The accumulation of the fusion protein reached up to 1.1% of TSP in mature leaves under the normal illumination even in highly heteroplasmic plants (FIG. 4b). The observed expression levels were higher when compared to other plant systems expressing TB vaccine antigens (Zelada et al. 2006).

### Example 7

### Quantification of CTB-ESAT6 Fusion Protein in Transplastomic Lettuce Plants by Densitometry

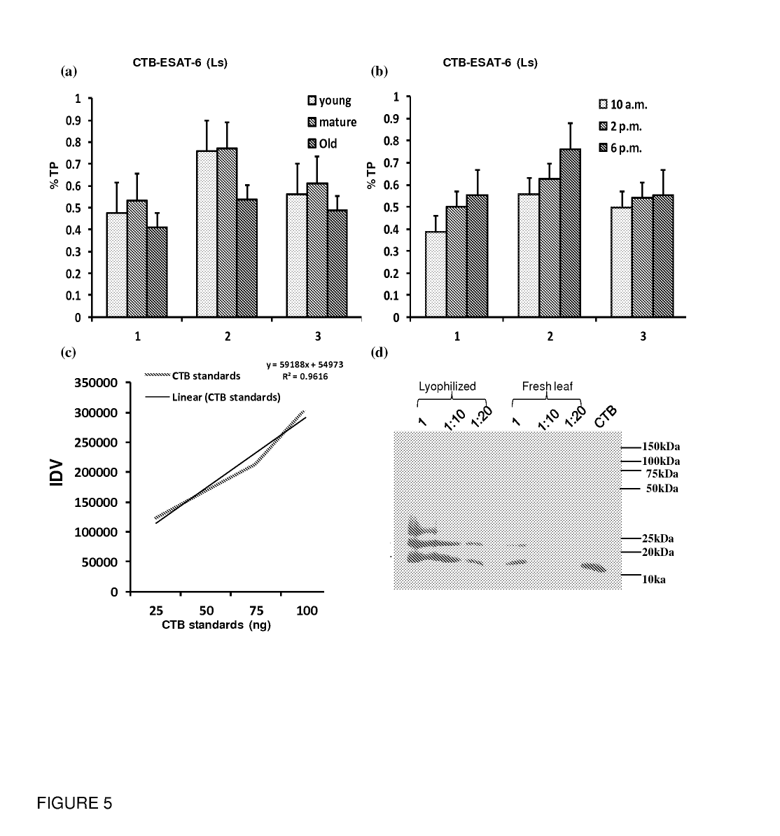

Immunoblots of different extraction fractions of CTB-ESAT6 lettuce transplastomic plants indicated the presence of fusion protein in both supernatant and pellet (FIG. 3f). Quantification of CTB-ESAT6-Ls fusion protein by ELISA couldn't be correlated with western analysis. Therefore, CTB-ESAT6-Ls expression levels were determined on western blots by comparing homogenate fraction with known quantities of purified CTB and analyzing them by spot densitometry. Linearity of the standard curve was established using 25, 50 and 75 ng of purified CTB, enabling the estimation of CTB-ESAT6 expression (FIG. 5c). In lettuce, CTB-ESAT6 accumulated up to 0.75% of total leaf protein (FIG. 5a). Mature leaves showed highest expression followed by young leaves (FIG. 5a). Old leaves showed lowest level of expression probably due to senescence and proteolytic activity. Similarly different time points of harvest were analyzed using densitometry. We observed an increasing trend from morning to evening with highest expression levels of fusion protein at 6 PM (FIG. 5b). One of the limitations in oral delivery studies is the low level of expression of protein. We performed lyophilization of CTB-ESAT6 expressing lettuce leaves for stable storage and discovered that the amount of antigen per gram of leaf material increased. After lyophilization, leaves were reduced to 5-8% of their fresh weight thereby increasing the CTB-ESAT6 antigenic content per gram of leaf material. Analysis of lyophilized material on immunoblots with anti-CTB antibody revealed same protein bands as obtained with fresh leaf material (FIG. 5d). There was a 22 fold increase in antigenic content per gram in lyophilized leaves when compared to fresh leaves.

### Example 8

### Affinity of Plant-Derived CTB-ESAT6 for GM1-Ganglioside Receptor

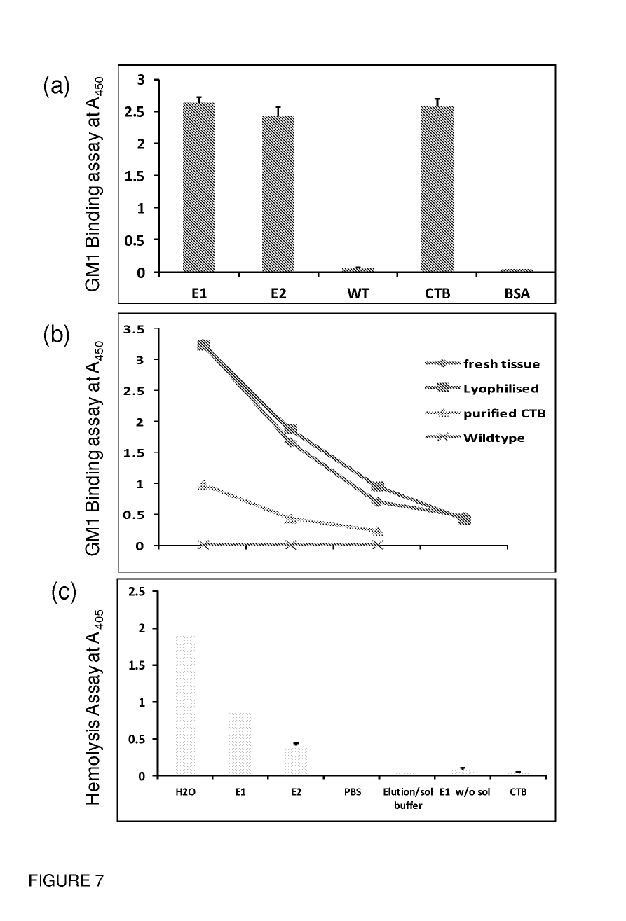

To evaluate whether CTB-ESAT6 fusion protein produced in lettuce retained its biological function of binding to the GM1 receptor, we performed GM1-binding ELISA assay. A pentameric structure of CTB protein is required for binding to its receptor GM1-ganglioside in vivo (Tsuji et al. 1995; de Haan et al. 1998). CTB-ESAT6 plants along with purified CTB protein showed strong binding affinity to GM-1 (FIGS. 7a & b). Untransformed plants and bovine serum albumin (BSA) didn't show binding to the GM1 receptor (FIGS. 7a & b). Effect of lyophilization on the binding ability of CTB-ESAT6 fusion protein was also tested with GM-1 ELISA binding assay. An extract from CTB-ESAT6 lyophilized lettuce leaves effectively bound to the GM1-ganglioside receptors (FIG. 7b). Further, serial dilution of extract from fresh, lyophilized leaves and purified CTB showed decrease in absorbance accordingly (FIG. 7b). GM1 binding of CTB-ESAT6 fusion protein from lyophilized and fresh lettuce leaves indicates that the CTB in the fusion protein has retained its native pentameric structure and has not been disrupted by its fusion to ESAT-6 or by lyophilization process. Binding to GM1 receptor is essential for antigen uptake in the gut.

### Example 9

### Detection of Pore Formation in Red Blood Cell Membranes by Purified CTB-ESAT6 Protein Using Hemolysis Assay

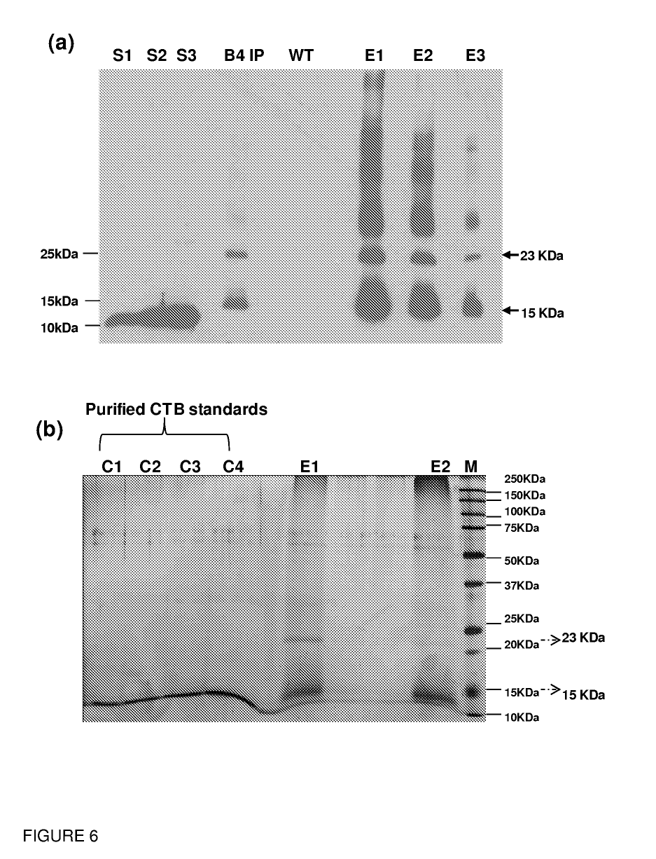

Lettuce expressing CTB-ESAT6 protein is an oral edible vaccine for TB, therefore there is no need to perform purification of antigens. Hence, a tag such as histidine was not incorporated in the coding sequence. To perform hemolysis assay purification of CTB-ESAT6 fusion protein is necessary. Therefore, we purified CTB-ESAT6 fusion protein from lettuce plants using immunoaffinity purification with CTB antibody. Western blot analysis of purified protein detected multiple bands corresponding to monomeric 23 kDa, cleaved 15 kDa and aggregates or multimers of >23 kDa molecular weight (FIG. 6a). Based on densitometry analysis, up to 40 μg/ml of 80% pure CTB-ESAT6 was obtained and this concentration was further confirmed by ELISA. Silver staining of purified protein showed two protein bands of ˜23 kDa and ˜15 kDa (FIG. 6b). Presence of ˜15 kDa protein band showed that the cleavage occurred in the ESAT6 protein. Purified CTB protein showed band at their corresponding size (FIG. 6b). Protein band corresponding to CTB-antibody was not detected in silver staining as CTB-antibody was cross linked on to the protein A bead support to prevent antibody being co eluted with the antigen. The purified CTB-ESAT6 protein was used for hemolysis assay.

MTB uses ESX-1 secretion system to export virulence proteins during infection. ESAT-6 is one of the secreted proteins in ESX-1 system and has been reported to play a role in the escape of Mycobacterium from the phagolysosome (van der Wel et al. 2007) by membrane pore formation (Smith et al. 2008). Purified ESAT-6 has been proven to cause dose dependent hemolysis in red blood cells by membrane pore formation (Smith et al. 2008). The red blood cell lysis by pore forming proteins occurs by osmotic shock. The hemolytic effect of plant-derived partially purified ESAT6 was investigated on red blood cell membranes. Hemolysis was measured by the absorbance (O.D.) of the red blood cell supernatant which contains hemoglobin. Partially purified CTB-ESAT6 when solubilized resulted in dose dependent hemolysis. CTB-ESAT6 formed aggregates in its native form and hence was solubilized to dissociate the oligomeric protein into its monomer form. Purified protein without solubilisation did not cause hemolysis at a concentration of 40 μg/ml (FIG. 7c). After solubilisation, CTB-ESAT6 at 40 μg/ml protein concentration caused partial hemolysis of red blood cells with an absorbance of 0.85. Decrease in absorbance to 0.4 was observed when the protein was diluted two fold (FIG. 7c). This indicated that the fusion did not modify ESAT-6 protein ability to form pores and lysed red blood cell membranes in a dose-dependent manner. Red blood cells incubated with water resulted in complete lysis (absorbance of 1.9) as the cells swell and burst due to movement of water into cells. Red blood cells incubated in PBS solution were intact and therefore no absorbance was detected (FIG. 7c). Complete hemolysis of red blood cell membranes was not accomplished with solubilized purified CTB-ESAT6 protein as higher concentrations (>40 μg/ml) could not be achieved by affinity purification.

### Discussion Related to Examples

Recent developments in genetic engineering have revealed enormous potential in plant chloroplasts such as high expression, cost-effectiveness, scalability and safety in production systems for recombinant biopharmaceuticals and vaccines (Daniell 2006; Yusibov et al. 2008; Daniell et al. 2009). It is believed that this is the first report of expression of TB vaccine antigens in chloroplasts. In this study, ESAT-6 and MTB72F, two attractive candidate antigens for TB vaccine development, were expressed in chloroplasts. Results exhibit an efficient and stable expression of the recombinant fusion protein CTB-ESAT6 and CTB-MTB72F in chloroplasts. The main factors that are essential for practical development of oral plant vaccines are sufficient expression level of antigens in the system used, adequate amount of dosage (leaf material) required for immunization, stability and storage of vaccines apart from efficacy. Chloroplast expression system proved to be successful for production of TB vaccine antigens to achieve all the parameters mentioned above.

In case of tobacco plants, the level of production of the CTB-ESAT6 reached up to 7.5% of the total soluble protein (TSP) in the mature transgenic tobacco leaves under normal illumination. This is 7-15 fold higher than that achieved in tobacco via transient expression (Zelada et al. 2006). The recombinant CTB-MTB72F accumulated up to 1.1% of TSP which could be due to heteroplasmy observed in transplastomic plants. Homoplasmy in CTB-MTB72F transgenic lines was not achieved even after three additional rounds of selection. This may be due to the toxic effect of improperly folded MTB72F as GPGP alone might not be sufficient to prevent steric hindrance. Homoplasmy was also not achieved in tobacco plants expressing CTB and factor IX fusion protein with GPGP hinge region in between whereas addition of the furin cleavage site helped in achieving homoplasmy and enhanced its accumulation 20-fold (Verma et al., 2010). CTB-MTB72F was developed in LAMD, a low nicotine variety of tobacco that is a suitable tobacco system for oral delivery of vaccine antigens. The CTB-ESAT6 lettuce transplastomic plants have modest expression levels of 0.75% of total leaf protein (TP). In comparison to tobacco system, lettuce has shown lower expression levels with other antigens (Ruhlman et al. 2010). The variation in expression levels of recombinant proteins can be due to many factors including nature of protein, plant system, environmental conditions, protein stability in chloroplasts and regulatory elements present in expression cassette (Scotti et al. 2011). Since tobacco system expressed higher levels of CTB-ESAT6, this variation could be due to protein stability and production in chloroplasts of lettuce.

The results provided herein indicated that approximately 950 μg of CTB-ESAT6 protein can be obtained per gram fresh weight of mature tobacco leaves under normal illumination. Accordingly, 80 mg of CTB-ESAT6 can be obtained from a single tobacco plant and a total of 1.92 kg can be produced from an acre of land based on three cuttings in a year. For some commercial tobacco cultivars whose yield are almost 20 fold more than that of experimental cultivar Petite Havana (Cramer et al. 1999), it is expected to produce more vaccine antigens at a great low cost of vaccination for a larger population. The CTB-ESAT6 lettuce plants expressed 11.2 μg/g of antigen (fresh weight mature leaf) whereas lyophilization increased the yield 22 fold as the antigenic content built up to 249 μg/g of lyophilized leaves. Hence large amount of transgenic protein is available for oral delivery. In human trials for TB subunit vaccine ESAT-6, 50 μg of vaccine antigen was injected intramuscularly (van Dissel et al. 2010). So if 50 μg were to be orally fed, based on quantification of antigenic content, only 200 mg of lyophilized material would be needed. Lyophilized lettuce expressing hepatitis B surface antigen has been successfully used in orally delivered plant vaccine animal studies (Pniewski et al. 2011). Therefore, all in vitro functional studies were carried out with lettuce plants.

Lettuce (Lactuca sativa) was chosen as an alternative to tobacco for expression of TB vaccine antigens due to its many advantages such as it being an edible crop, its leafy nature and commercial importance. The lettuce CTB-ESAT6 plants showed modest expression levels which is believed can be remedied with lyophilization, a process of freeze drying. Since lettuce has high water content (95%), it can be freeze dried to a greater magnitude than tobacco, potato etc. Since it is a leafy vegetable, more antigen could be concentrated by lyophilization of leaf tissue. The CTB-ESAT6 protein was stable in lyophilized material stored at room temperature for six months. This stability of antigens in plant tissue could help in eliminating cold chain during storage and distribution required for conventional vaccines.

Functional analysis of CTB-ESAT6 fusion protein was performed. The ability of CTB to form pentamers allows it to bind to GM-1 ganglioside receptors and gives it the advantage of increased antigen uptake. GM-1 ELISA binding assay revealed the ability of CTB-ESAT6-Ls fusion protein to form pentamers and bind to the GM-1 receptors. ESAT-6 is a secreted protein that is observed in early mycobacterial infections. Its activity has been characterized as a cytolysin that can disrupt lipid bilayers (Hsu et al. 2003). Hemolysis assay established the ability of partially purified CTB-ESAT6 to create partial lysis of red blood cell membranes. Plant derived CTB-ESAT6 has been shown to retain its biologically activity and has potential to be an effective oral vaccine.

Production of an oral subunit vaccine against tuberculosis in chloroplast is a promising strategy to overcome the cost constraints such as production, purification, processing, cold storage, transportation and delivery linked to large vaccination campaigns, especially with the increasing TB incidence in some developing countries where there is a dire need of such vaccines. It is estimated that the costs associated with production and delivery of the recombinant proteins in bacterial, insects or mammalian cells is much higher when compared to plants (Chebolu et al. 2009). Easy and unlimited scalability of protein production and absence of the viral contamination can make plant-derived biologics economical and safer for large-scale production (Goldstein et al. 2004; Yusibov et al. 2008).

For oral delivery analysis of this TB vaccine antigen expressed in chloroplast, animal studies are performed to test immunogenicity. Lettuce, an edible crop plant and LAMD, low nicotine tobacco variety are ideal systems for oral delivery of CTB-ESAT6 and CTB-MTB72F respectively. Multistage vaccines will be significant against a complex disease such as tuberculosis. So constructing a vaccine with multiple antigens that can target early and late stage tuberculosis can pave the way for future oral TB vaccine.

### Materials and Methods Related to Examples

Construction of pLD-CTB-ESAT6, pLS-CTB-ESAT6 and pLD-CTB-MTB72F Vectors for Chloroplast Transformation

The ESAT-6 sequence was amplified using sequence-specific restriction-site flanking primers and Mycobacterium tuberculosis genomic DNA as template. The PCR product was then cloned into the pCR BluntII Topo vector (Invitrogen) and sequenced to check any errors. Following SmaI/XbaI digestion, the ESAT-6 gene was ligated into the pLD Ctv 5CP chloroplast transformation vector (Ruhlman et al. 2007) to create pLD-CTB-ESAT6. The CTB sequence was amplified using sequence specific primers and pLD-5′-UTR-CTBPins (Ruhlman et al., 2007) vector as the template. Mtb72F (Skeiky et al., 2004) was generated by amplifying each individual fragment (Mtb32c¹⁹²⁻³²³, SmaI at 5′ end and BamHI at 3′ end; Mtb39, BamHI at 5′ end and EcoRI at 3′ end; and Mtb32n¹⁻¹⁹⁵, EcoRI at 5′ end and HindIII at 3′ end) from Mycobacterium tuberculosis genomic DNA using sequence specific primers and sequentially linking in tandem the 14-kDa C-terminal fragment of mtb32 to the full-length fragment of mtb39, followed by the 20-kDa N-terminal portion of mtb32. Further, the sequences were confirmed to verify any errors and assembled as CTB-MTB72F into the pBSSK+ (Stratagene, La Jolla, Calif., USA) vector. The CTB-MTB72F was then sub cloned into the tobacco chloroplast transformation vector to obtain pLD-CTB-MTB72F.

Lettuce flanking sequence vector (pLSLF) was designed to integrate the transgene into transcriptionally active spacer region between trnI and trnA genes as explained previously (Ruhlman et al. 2007; Verma et al. 2008). The gene cassette comprising promoter Prrn and rbcI from lettuce chloroplast genome and selectable marker aadA gene was built into pBSSK+ vector and cloned into pLSLF. pLD-5′-UTR-CTBPins (Ruhlman et al. 2007) vector was used as the template to amplify the CTB sequence. Further, the ESAT-6 sequence was amplified using sequence-specific restriction-site flanking primers and Mycobacterium tuberculosis genomic DNA as the template. The CTB-ESAT6 gene was ligated in to pLSLF to create pLS-CTB-ESAT6 vector along with psbA promoter and 5′ and 3′ UTR from lettuce regulatory regions. To facilitate proper folding of protein, both fusions (CTB-ESAT6 and CTB-MTB72F) had the GPGP hinge in between fusion proteins for reducing the steric hindrance.

### Bombardment and Selection of Transgenic Plants

Chloroplast transformation including bombardment and regeneration was carried out as described previously (Kumar et al. 2004; Verma et al. 2008). In brief, sterile fully expanded leaves placed on MS medium with abaxial side up for tobacco (Nicotiana tabacum var Petite Havana), LAMD and adaxial side up for lettuce (Lactuca sativa) were bombarded with gold particles coated with plasmid DNA of pLD-CTB-ESAT6, pLD-CTB-MTB72F and pLS-CTB-ESAT6 respectively using the biolistic device PDS1000/He (Bio-Rad). After incubation at 25° C. in the dark for 2 days, the leaves were cut into small (˜5 mm²) pieces and placed on the regeneration medium of plants (RMOP) containing spectinomycin dihydrochloride 500 mg/l (for Petite Havana), 200 mg/l (for LAMD) and modified LR regeneration medium of lettuce prepared with spectinomycin dihydrochloride 50 mg/l (for lettuce) with bombarded side facing the medium. (Ruhlman et al., 2007) The transgenic shoots appeared after about 4-8 weeks and were screened for transgene integration by PCR as described below. PCR positive shoots underwent an additional selection on their corresponding regeneration medium and were rooted in half-strength MS medium containing spectinomycin of concentrations mentioned earlier. Rooted plants were transferred to Jiffy peat pots and placed in incubator for acclimatization. After considerable growth, plants were moved to green house for maturation and seed production. In order to confirm maternal inheritance, seeds harvested from the CTB-ESAT6 plants transplastomic plants were germinated on ½ MS salt supplemented with spectinomycin (100 mg/l for lettuce). Sterilization of seeds was performed with 1.5% bleach, followed by thorough rinsing in distilled water. Seeds from untransformed plants were also grown in the same plate. The growth of the plants was observed after 10 days.

Confirmation of Transgene Integration into the Chloroplast Genome by PCR and Southern Blot Analysis

To confirm the transgene cassette(s) integration into the chloroplast genome, genomic DNA was extracted from leaf tissues of spectinomycin resistant and wild-type untransformed plants using Qiagen DNeasy Plant Mini Kit (Qiagen, Valencia, Calif.). PCR analysis was performed using the primer pair 3P (5′-AAAACCCGTCCTCAGTTCGGATTGC-3′) for tobacco or 16SF (5′-CAGCAGCCGCGGTAATACAGAGGA-3′) for lettuce and 3M (5′-CCGCGTTGTTTCATCAAGCCTTACG-3′). Additionally, to validate the integration of transgene of interest, another primer pair 5P (5′-CTGTAGAAGTCACCATTGTTGTGC-3′) and 2M (5′-TGACTGCCCAACCTGAGAGCGGACA-3′) was used. Southern blot analysis was carried out to confirm site specific integration and to determine homoplasmy as described previously (Kumar et al. 2004). Regenerated chloroplast transgenic lines were analyzed to determine whether homoplasmy was obtained with all the copies of the chloroplast genome containing stably integrated transgene. In brief, 2-5 μg of genomic DNA of both tobacco and lettuce were digested completely with HindIII enzyme and run on a 0.7% (w/v) tris-acetate EDTA (TAE) agarose gel and transferred to a nylon membrane (N⁺-Bond, Amersham Biosciences, USA) by capillary action. The P³² labeled flanking sequence probe (˜0.81 kb, FIG. 1c) was generated by digesting chloroplast vector pUC-CT with BglII and BamHI (Lee et al. 2003) for southern blot analysis of tobacco plants. Pre-hybridization and hybridization were carried out using hybridization solution (Stratagene QUICK-HYB, La Jolla, Calif.) as described in company protocol. The membrane was exposed to X-ray film in a cassette

in −80° C. for 2 days.

### Detection of Fusion Protein Using Western Blot Analysis

Transformed and untransformed leaves (˜100 mg) were ground in liquid nitrogen with a mortar and pestle followed by extraction with a mechanical pestle in 200 μl of extraction buffer (200 mM Tris-HCl, pH 8.0, 100 mM NaCl, 0.1% SDS, 400 mM sucrose, 0.05% Tween 20, 2 mM PMSF and proteinase inhibitor cocktail (Roche)). The leaf extracts were then centrifuged for 5 min at 10,000 rpm to separate out the supernatant and pellet the insoluble plant material. The pellet was resuspended in buffer and sonicated in ice for 10 s pulse for a min. Bradford assay was performed to detect total protein concentration using Protein Assay Dye Reagent Concentrate (Bio-Rad). Standard curve for this assay was generated with Bovine serum Albumin (BSA) with dilutions ranging from 0.8 mg/ml to 0.025 mg/ml. All samples were loaded in duplicate. Protein Assay dye was dilutes and absorbance was measured at 595 nm. Homogenate, supernatant and pellet fractions were boiled for 5 min in sample buffer (0.5M Tris-HCl, 25% glycerol, 10% SDS, 0.5% Bromophenol blue and β-mercapto ethanol) and separated by sodium dodecyl sulfate-polyacrylamide gel electrophoresis (SDS-PAGE) (Bio-Rad). The separated proteins were then transferred on to a nitrocellulose membrane in a transfer cassette (Bio-Rad) at 85V for 1 hour. After blocking with phosphate-buffered saline (PBS), 0.1% Tween 20, 3% milk powder (PTM), the membrane was incubated with anti-CTB primary antibody (1:4000, Sigma, St. Louis, Mo., USA) diluted in PTM followed by 1:5000 horseradish peroxidase (HRP)-conjugated goat anti-rabbit secondary antibody (Southern biotech, Birmingham, Ala., USA) for 1 hour 30 minutes. A Super Signal West Pico HRP Substrate Kit (Pierce, Rockford, Ill., USA) was used for autoradiographic detection.

### Enzyme Linked ImmunoSorbent Assay

To quantify the expression of the fusion protein, the enzyme linked immunosorbent assay (ELISA) was performed. Approximately one hundred milligram (mg) of the leaf samples at different developmental stages (young, mature and old) or at different time points (10 a.m., 12 p.m. and 6 p.m.) during the day were collected from plants exposed to regular lighting pattern (16 h light and 8 h dark). The extraction buffer explained above was used to isolate total leaf protein for this assay. The CTB (sigma) protein standards and tested samples were diluted in the coating buffer (15 mM Na₂CO₃, 35 mM NaHCO₃, 3 mM NaN₃, pH 9.6) with the concentration from 50 to 1000 ng/mL and coated on to a 96 well ELISA plate overnight at 4° C. Blocking was performed with PTM for 1 hour. The anti-CTB primary antibody was used at 1:4000 dilution for 1 hour. Washes were performed with 1×PBS, 0.1% tween (PBST) thrice followed by washes with distilled water. HRP conjugated goat anti-rabbit secondary antibody was used at 1:5000 dilution for 1 hour. Washes were performed as mentioned above. Wells were then loaded with 100 μl of 3,3,5,5-tetramethylbenzidine (TMB; American Qualex) substrate and incubated for 10 to 15 min at room temperature. The reaction was terminated by adding 50 μl of 2N H₂SO₄ per well, and the plate was read on a plate reader (Dynex Technologies) at 450 nm.

### Densitometry Analysis

To quantify the expression of fusion protein, in case of the lettuce plant, we performed densitometric analysis on the immunoblots with CTB antibody. Known concentrations of purified CTB (Sigma) were used as standards (25, 50, 75 and 100 ng) to create a standard curve. Total protein concentration was measured using Bradford assay (Bio-Rad). Fusion protein was loaded in different concentrations of total protein and their integrated Density values (IDV) was measured using Alpha imager 2000 and analyzed using Alphaease software. The percentage of fusion protein (% Total leaf protein) and amount of transgenic protein (μg/g) is calculated based on the formula published earlier (Verma et al. 2008).

### GM1—Ganglioside Receptor Binding Assay:

GM-1 ganglioside (Sigma G-7641) and BSA (control) was coated at a concentration 3 μg/ml in bicarbonate buffer (15 mM Na2CO3, 35 mM NaHCO3, pH 9.6) on to a 96 well plate at 4° C. overnight. Washing was performed thrice with 1×PBST and water. The wells were then blocked with 200 μl of phosphate-buffered saline (PBS)—0.1% Tween—3% milk powder (PTM) for 2 hours at 37° C. followed by washing thrice with 1×PBST and water. CTB protein standards, Untransformed plant leaf TSP and transformed leaf TSP diluted in ELISA coating buffer were coated on to the plate in different concentrations and incubated for 2 hours at 37° C. The plate was washed again as stated above and incubated with anti-CTB primary antibody (1:3000 dilution) for 1 hr at 37° C. Further, the plate was incubated with secondary HRP-conjugated goat anti-rabbit IgG in 1:4000 dilution. Following washing with 1×PBST and water thrice, 100 μl of 3,3,5,5-tetramethylbenzidine substrate was added and incubated for 10 to 15 min at room temperature. The reaction was terminated by adding 50 μl of 2 N H₂SO₄ per well and the absorbance was read on a plate reader at 450 nm.

### Affinity Purification:

Total leaf protein of CTB-ESAT6 was extracted with 2×PBS, 0.1% Tween—20, pH 8, proteinase inhibitors and sonicated (Sonicator 3000 Misonix) for 1 min in ice. Supernatant was obtained after centrifuging for 5 min at 2000 rpm. Pre-clearing of the supernatant was performed with 100 mg of Protein A Sepharose CL-4B (GE Healthcare) beads along with protease inhibitors (Roche) overnight at 4° C. Rabbit anti-cholera toxin B subunit polyclonal antibody (Abcam, ab34992) was coated on to washed protein A beads in a ratio of 50 μg:200 μl in 1×PBS overnight at 4° C. for antibody binding to beads. Washes were performed with 1×PBS followed by 0.1M sodium borate buffer (pH 9). 20 mM Dimethyl pimelimidate—DMP (sigma, D8388) cross linker in sodium borate buffer was added to the bead-antibody mixture and incubated for 30 min at room temperature twice. Washes were continued with 50 mM glycine pH 2.5 followed by 1×PBS. Cross linked antibody was added to the precleared supernatant and incubated overnight at 4° C. Elutions were performed with 100 mM glycine buffer, pH 2.5 in 100 μl volume with 10 μl of 1M Tris-HCl pH 9 for neutralization at room temperature. All Elution fractions were analyzed by Bradford assay (Bio-Rad) and nanodrop spectrophotometer via absorbance at 280 nm for total protein concentration in mg/ml. Silver staining was performed to detect purity %.

### Silver Staining:

SDS-PAGE gel (12%) was fixed using a fixative (50% methanol, 12% acetic acid) overnight at 4° C. and washed twice with 50% ethanol. The gel was pretreated with 0.02% sodium thiosulphate solution (Na₂S₂O₃) for 1 min and then washed thrice in distilled water for 1 min each. Gel was stained with Silver nitrate solution (0.2% silver nitrate in formalin) for 20 min. The gel was rinsed twice in distilled water for 1 min and developer solution (2% Na₂CO₃, 0.0004% Na₂S₂O₃, formalin) was added. Gel was shaken to observe bands in developer solution. Developer solution was replaced after 5 min and reaction was stopped with 1% acetic acid as soon as protein bands were observed.

### Lyophilization:

CTB-ESAT-6 lettuce leaves were frozen in liquid nitrogen and then lyophilized in Freezone Benchtop Freeze Dry Systems (Labconco) in vacuum for 2 days at −50° C. at 0.036 mBar. After lyophilization, they were stored at room temperature in vacuum for few days. The samples were ground to fine powder in waring blender and further testing was performed. Lyophilized leaf tissue was normalized with fresh leaf tissue based on its dry weight. Immunoblots were performed with 5 g of Lyophilized tissue and 100 g of fresh tissue since we observe 95% decrease in weight due to removal of water content. We also performed GM-1 ELISA binding assay to confirm stability of fusion protein in Lyophilized tissue.

### Hemolysis Assay:

To test the hemolytic activity of the ESAT-6 protein, hemolysis assay was performed with affinity purified CTB-ESAT6 fusion protein. Pore formation in red blood cell membranes can be measured by hemolysis assay as shown previously (Smith et al. 2008). Affinity purified CTB-ESAT-6 was solubilized with 0.1 M KCl—HCl for 1 min followed by neutralizing with 50 mM Tris-HCl, pH 8 as described earlier (Ruddock et al. 1996; Tinker et al. 2003). After solubilization, CTB-ESAT6 fusion protein was mixed with sheep red blood cells (1×10⁹ cells) in a micro centrifuge tube in the ratio 1:2 and allowed to incubate at 32° C. for 2 hours. As controls, non solubilized CTB-ESAT6, distilled water and PBS were incubated with sheep red blood cells in the same ratio and under same conditions. The cells were then resuspended and centrifuged for about 7 min at 4000 rpm. Supernatants were loaded on to 96 well plate and absorbance of the supernatants was read at 405 nm.

### REFERENCES

Agger, E. M. and P. Andersen (2001). “Tuberculosis subunit vaccine development: on the role of interferon-gamma.” Vaccine 19(17-19): 2298-2302.
Andersen, P. (2007). “Tuberculosis vaccines [mdash] an update.” Nat Rev Micro 5(7): 484-487.
Andersen, P., A. B. Andersen, et al. (1995). “Recall of long-lived immunity to Mycobacterium tuberculosis infection in mice.” J Immunol 154(7): 3359-3372.
Arlen, P. A., M. Singleton, et al. (2008). “Effective plague vaccination via oral delivery of plant cells expressing F1-V antigens in chloroplasts.” Infect Immun 76(8): 3640-3650.
Bally, J., M. Nadai, et al. (2009). “Plant physiological adaptations to the massive foreign protein synthesis occurring in recombinant chloroplasts.” Plant Physiol 150(3): 1474-1481.
Bally, J., E. Paget, et al. (2008). “Both the stroma and thylakoid lumen of tobacco chloroplasts are competent for the formation of disulphide bonds in recombinant proteins.” Plant Biotechnology Journal 6(1): 46-61.
Brandt, L., M. Elhay, et al. (2000). “ESAT-6 Subunit Vaccination against Mycobacterium tuberculosis.” Infect. Immun. 68(2): 791-795.
Brewer, T. F. and G. A. Colditz (1995). “Relationship Between Bacille Calmette-Guerin (BCG) Strains and the Efficacy of BCG Vaccine in the Prevention of Tuberculosis.” Clinical Infectious Diseases 20(1): 126-135.
Brodin, P., I. Rosenkrands, et al. (2004). “ESAT-6 proteins: protective antigens and virulence factors?” Trends in Microbiology 12(11): 500-508.
Chebolu, S. and H. Daniell (2009). “Chloroplast-derived vaccine antigens and biopharmaceuticals: expression, folding, assembly and functionality.” Curr Top Microbiol Immunol 332: 33-54.
Colditz, G. A., T. F. Brewer, et al. (1994). “Efficacy of BCG Vaccine in the Prevention of Tuberculosis.” JAMA: The Journal of the American Medical Association 271(9): 698-702.
Coler, R. N., D. C. Dillon, et al. (2009). “Identification of Mycobacterium tuberculosis vaccine candidates using human CD4+ T-cells expression cloning.” Vaccine 27(2): 223-233.
Cramer, C. L., J. G. Boothe, et al. (1999). “Transgenic plants for therapeutic proteins: linking upstream and downstream strategies.” Curr Top Microbiol Immunol 240: 95-118.
Daniell, H. (2002). “Molecular strategies for gene containment in transgenic crops.” Nat Biotech 20(6): 581-586.
Daniell, H. (2006). “Production of biopharmaceuticals and vaccines in plants via the chloroplast genome.” Biotechnology Journal 1(10): 1071-1079.
Daniell, H., S.-B. Lee, et al. (2001). “Expression of the native cholera toxin B subunit gene and assembly as functional oligomers in transgenic tobacco chloroplasts.” Journal of Molecular Biology 311(5): 1001-1009.
Daniell, H., N. D. Singh, et al. (2009). “Plant-made vaccine antigens and biopharmaceuticals.” Trends Plant Sci 14(12): 669-679.
Davoodi-Semiromi, A., M. Schreiber, et al. (2010). “Chloroplast-derived vaccine antigens confer dual immunity against cholera and malaria by oral or injectable delivery.” Plant Biotechnology Journal 8(2): 223-242.
de Haan, L., W. R. Verweij, et al. (1998). “Role of GM1 binding in the mucosal immunogenicity and adjuvant activity of the Escherichia coli heat-labile enterotoxin and its B subunit.” Immunology 94(3): 424-430.
Derrick, S. C., A. L. Yang, et al. (2004). “A polyvalent DNA vaccine expressing an ESAT6-Ag85B fusion protein protects mice against a primary infection with Mycobacterium tuberculosis and boosts BCG-induced protective immunity.” Vaccine 23(6): 780-788.
Dhingra, A. and H. Daniell (2006). “Chloroplast genetic engineering via organogenesis or somatic embryogenesis.” Methods Mol Biol 323: 245-262.
Doherty, T. M., J. Dietrich, et al. (2007). “Tuberculosis subunit vaccines: from basic science to clinical testing.” Expert Opinion on Biological Therapy 7(10): 1539-1549.
Dorokhov, Y. L., A. A. Sheveleva, et al. (2007). “Superexpression of tuberculosis antigens in plant leaves.” Tuberculosis 87(3): 218-224.
Fernandez-San Millan, A., A. Mingo-Castel, et al. (2003). “A chloroplast transgenic approach to hyper-express and purify Human Serum Albumin, a protein highly susceptible to proteolytic degradation.” Plant Biotechnol J 1(2): 71-79.
Glenz, K., B. Bouchon, et al. (2006). “Production of a recombinant bacterial lipoprotein in higher plant chloroplasts.” Nat Biotech 24(1): 76-77.
Goldstein, D. A. and J. A. Thomas (2004). “Biopharmaceuticals derived from genetically modified plants.” QJM 97(11): 705-716.
Hamasur, B., M. Haile, et al. (2003). “Mycobacterium tuberculosis arabinomannan-protein conjugates protect against tuberculosis.” Vaccine 21(25-26): 4081-4093.
Hsu, T., S. M. Hingley-Wilson, et al. (2003). “The primary mechanism of attenuation of bacillus Calmette-Guérin is a loss of secreted lytic function required for invasion of lung interstitial tissue.” Proceedings of the National Academy of Sciences 100(21): 12420-12425.
Koya, V., M. Moayeri, et al. (2005). “Plant-Based Vaccine: Mice Immunized with Chloroplast-Derived Anthrax Protective Antigen Survive Anthrax Lethal Toxin Challenge.” Infect. Immun. 73(12): 8266-8274.
Kumar, M., N. Meenakshi, et al. (2010). “Immune response to Mycobacterium tuberculosis specific antigen ESAT-6 among south Indians.” Tuberculosis 90(1): 60-69.
Kumar, S., A. Dhingra, et al. (2004). “Plastid-Expressed Betaine Aldehyde Dehydrogenase Gene in Carrot Cultured Cells, Roots, and Leaves Confers Enhanced Salt Tolerance.” Plant Physiology 136(1): 2843-2854.
Langridge, W., B. Denes, et al. (2010). “Cholera toxin B subunit modulation of mucosal vaccines for infectious and autoimmune diseases.” Curr Opin Investig Drugs 11(8): 919-928.
Lee, S. B., H. B. Kwon, et al. (2003). “Accumulation of trehalose within transgenic chloroplasts confers drought tolerance.” Molecular breeding: new strategies in plant improvement 11(1): 1-13.
Ma, J. K., P. M. Drake, et al. (2003). “The production of recombinant pharmaceutical proteins in plants.” Nat Rev Genet 4(10): 794-805.
Pniewski, T., J. Kapusta, et al. (2011). “Low-dose oral immunization with lyophilized tissue of herbicide-resistant lettuce expressing hepatitis B surface antigen for prototype plant-derived vaccine tablet formulation.” J Appl Genet 52(2): 125-136.
Rigano, M. M., M. L. Alvarez, et al. (2004). “Production of a fusion protein consisting of the enterotoxigenic Escherichia coli heat-labile toxin B subunit and a tuberculosis antigen in Arabidopsis thaliana.” Plant Cell Rep 22(7): 502-508.
Ruddock, L. W., J. J. Coen, et al. (1996). “Assembly of the B subunit pentamer of Escherichia coli heat-labile enterotoxin. Kinetics and molecular basis of rate-limiting steps in vitro.” J Biol Chem 271(32): 19118-19123.
Ruhlman, T., R. Ahangari, et al. (2007). “Expression of cholera toxin B-proinsulin fusion protein in lettuce and tobacco chloroplasts—oral administration protects against development of insulitis in non-obese diabetic mice.” Plant Biotechnology Journal 5(4): 495-510.
Ruhlman, T., D. Verma, et al. (2010). “The role of heterologous chloroplast sequence elements in transgene integration and expression.” Plant Physiol 152(4): 2088-2104.
Scotti, N., M. M. Rigano, et al. (2011). “Production of foreign proteins using plastid transformation.” Biotechnol Adv.
Sereinig, S., M. Stukova, et al. (2006). “Influenza virus NS vectors expressing the mycobacterium tuberculosis ESAT-6 protein induce CD4+ Th1 immune response and protect animals against tuberculosis challenge.” Clin Vaccine Immunol 13(8): 898-904.
Skeiky, Y. A. W., M. R. Alderson, et al. (2004). “Differential Immune Responses and Protective Efficacy Induced by Components of a Tuberculosis Polyprotein Vaccine, Mtb72F, Delivered as Naked DNA or Recombinant Protein.” The Journal of Immunology 172(12): 7618-7628.
Smith, J., J. Manoranjan, et al. (2008). “Evidence for Pore Formation in Host Cell Membranes by ESX-1-Secreted ESAT-6 and Its Role in Mycobacterium marinum Escape from the Vacuole.” Infect. Immun. 76(12): 5478-5487.
Stern, D. B. and W. Gruissem (1987). “Control of plastid gene expression: 3′ inverted repeats act as mRNA processing and stabilizing elements, but do not terminate transcription.” Cell 51(6): 1145-1157.
Tinker, J. K., J. L. Erbe, et al. (2003). “Cholera holotoxin assembly requires a hydrophobic domain at the A-B5 interface: mutational analysis and development of an in vitro assembly system.” Infect Immun 71(7): 4093-4101.
Triccas, J. A. (2010). “Recombinant BCG as a vaccine vehicle to protect against tuberculosis.” Bioengineered Bugs 1(2): 110-115.
Tsenova, L., R. Harbacheuski, et al. (2006). “Evaluation of the Mtb72F Polyprotein Vaccine in a Rabbit Model of Tuberculous Meningitis.” Infect. Immun. 74(4): 2392-2401.
Tsuji, T., K. Watanabe, et al. (1995). “Monomer of the B subunit of heat-labile enterotoxin from enterotoxigenic Escherichia coli has little ability to bind to GM1 ganglioside compared to its coligenoid.” Microbiol Immunol 39(10): 817-819.
van der Wel, N., D. Hava, et al. (2007). “M. tuberculosis and M. leprae translocate from the phagolysosome to the cytosol in myeloid cells.” Cell 129(7): 1287-1298.
van Dissel, J. T., S. M. Arend, et al. (2010). “Ag85B-ESAT-6 adjuvanted with IC31® promotes strong and long-lived Mycobacterium tuberculosis specific T cell responses in naïve human volunteers.” Vaccine 28(20): 3571-3581.
Verma, D., N. P. Samson, et al. (2008). “A protocol for expression of foreign genes in chloroplasts.” Nat Protoc 3(4): 739-758.
WHO (2010). Global Tuberculosis Control. WHO report 2010, World Health Organisation.
Xing, Z. and B. D. Lichty (2006). “Use of recombinant virus-vectored tuberculosis vaccines for respiratory mucosal immunization.” Tuberculosis 86(3-4): 211-217.
Yusibov, V. and S. Rabindran (2008). “Recent progress in the development of plant derived vaccines.” Expert Rev Vaccines 7(8): 1173-1183.
Zelada, A. M., G. Calamante, et al. (2006). “Expression of tuberculosis antigen ESAT-6 in Nicotiana tabacum using a potato virus X-based vector.” Tuberculosis 86(3-4): 263-267.

Unless otherwise defined, all technical and scientific terms used herein have the same meaning as commonly understood by one of ordinary skill in the art of molecular biology. Although methods and materials similar or equivalent to those described herein can be used in the practice or testing of the present invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and are not intended to be limiting.

Reference is made to standard textbooks of molecular biology that contain definitions and methods and means for carrying out basic techniques, encompassed by the present invention. See, for example, Maniatis et al., Molecular Cloning: A Laboratory Manual, Cold Spring Harbor Laboratory Press, New York (1982) and Sambrook et al., Molecular Cloning: A Laboratory Manual, Cold Spring Harbor Laboratory Press, New York (1989); Methods in Plant Molecular Biology, Maliga et al, Eds., Cold Spring Harbor Laboratory Press, New York (1995); Arabidopsis, Meyerowitz et al, Eds., Cold Spring Harbor Laboratory Press, New York (1994) and the various references cited therein. U.S. Patent Publication 20030009783 and 20060031964 are also cited for plant transformation techniques.

Finally, while various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims. The teachings of all patents and other references cited herein are incorporated herein by reference in their entirety to the extent they are not inconsistent with the teachings herein.

1-34. (canceled)

35. A plant plastid comprising a plastid genome transformed with a heterologous DNA coding sequence encoding at least one fusion protein selected from the group consisting of CTB-ESAT-6 and CTB-Mtb72F, said coding sequence being integrated into said plastid genome such that said CTB-ESAT-6 or CTB-Mtb72F is expressed in and present in said plastid.

36. An edible plant cell comprising the plastid of claim 35.

37. The plant cell of claim 36, present in a plant selected from the group consisting of lactuca sativa, apple, tomato and carrot.

38. An edible composition comprising the plant cell of claim 36.

39. A method of vaccinating a subject against TB comprising oral administration to said subject the composition of claim 38 comprising a CTB-ESAT-6 or CTB-Mtb72F polypeptide expressed in a chloroplast in a plant and, optionally, a plant remnant.

40. The method of claim 39, wherein said plant remnant is rubisco.

41. The method of claim 39, wherein said plant is Lactuca sativa, apple, tomato or carrot.

42. A stable plastid transformation and expression vector for expression of the heterologous DNA coding sequence of claim 35 in a plastid from Lactuca sativa, said vector comprising an expression cassette comprising, as operably linked components in the 5′ to the 3′ direction of translation, a promoter operative in said plastid, a selectable marker sequence, a heterologous polynucleotide sequence coding for CTB-ESAT-6 and or CTB-Mtb72F protein, a transcription termination sequence functional in said plastid, and flanking each side of the expression cassette, flanking DNA sequences which are homologous to a DNA sequence of the Lactuca sativa plastid genome, and suitable to effect homologous recombination between said vector and homologous sequences within said plastid genome.

43. The composition of claim 38, wherein said CTB-ESAT-6 or CTB-Mtb72F is bioencapsulated within said chloroplasts.

44. The vector of claim 42, wherein the selectable marker sequence is an antibiotic-free selectable marker.

45. A stably transformed plant which comprises a plastid stably transformed with the vector of claim 42 or the progeny thereof, including seeds.

46. The stably transformed plant of claim 45 wherein said chloroplasts are homoplasmic.

47. A process for producing a CTB-ESAT-6 and, or, CTB-Mtb72F polypeptide comprising:
a) integrating a plastid transformation vector according to claim 42 into the plastid genome of a Lactuca sativa plant cell;
b) growing said plant cell under conditions suitable for expression of said CTB-ESAT-6 or CTB-Mtb72F polypeptide; and
c) optionally homogenizing material of said stably transformed Lactuca sativa plant to produce homogenized material.

48. The method of claim 47, further comprising purifying CTB-ESAT-6 or CTB-Mtb72F from said homogenized material.

49. The method of claim 48, wherein said homogenized material is dried to produce a powder.

50. The method of claim 49, further comprising encapsulating said powder.

51. The composition of claim 38, which is freeze dried.

52. The composition of claim 51, wherein said CTB-ESAT-6 or CTB-Mtb72F, both retains therapeutic effectiveness when stored at room temperature for at least 2, 3, 4, 6, 7, 8, 9, 10, 11, 12, 13, 14, 15, 16, 17, 18, 19, 20, or 21 weeks.
